(19) United States
(12) Patent Application Publication
(10) Pub. No.: US 2013/0000280 A1
(43) Pub. Date: Jan. 3, 2013

(54) GAS MONITORING METHOD IMPLEMENTING SOOT CONCENTRATION DETECTION

(75) Inventor: Sergey KORENEV, Mundelein, IL (US)

(73) Assignee: Caterpillar, Inc.

(21) Appl. No.: 13/174,373

(22) Filed: Jun. 30, 2011

(51) Int. Cl. F01N 11/00; G01N 33/22

(52) U.S. Cl. 60/276; 73/23.31

(57) ABSTRACT

A method for detecting soot in a gas is disclosed. The method may include applying a voltage pulse to electrodes exposed to the gas, wherein the voltage pulse has a higher voltage amplitude than a breakdown voltage of the gas. The method may also include detecting breakdown of the gas, and determining a time to breakdown of the gas since application of the voltage pulse. The method may additionally include determining a concentration of particulate matter entrained in the gas based on the time to breakdown.

### TECHNICAL FIELD

The present disclosure is directed to a gas monitoring method, and more particularly to a gas monitoring method implementing soot concentration detection.

### BACKGROUND

Internal combustion engines, including diesel engines, gasoline engines, gaseous fuel-powered engines, and other engines known in the art exhaust a complex mixture of air pollutants. These air pollutants may include, among other things, solid particulate matter also known as particulates or soot. Due to increased awareness of the environment, exhaust emission standards have become more stringent, and the amount of particulate matter emitted from an engine may be regulated depending on the type of engine, size of engine, and/or class of engine.

One method that has been implemented by engine manufacturers to comply with the regulation of engine exhaust pollutants has been to detect a concentration of particulate matter within an exhaust gas, and then treat the gas through various filtering or trapping processes. One attempt to improve detection of particulate matter with a gas is described in U.S. Patent Application Publication No. 2010/0229632 of Tokuda (the '632 publication) that published on Sep. 16, 2010. In particular, the '632 publication discloses a device for detecting particulate matter in gas that includes a detection device body that has at least one through-hole that is formed at one end of the body, a high voltage electrode and a low voltage electrode that are buried in the wall of the body, a high voltage takeout lead terminal that is disposed on the surface of the body, a high voltage takeout lead terminal insulating member that is disposed to cover at least an area in which the lead terminal is disposed, and a detection device outer tube that is disposed to cover the lead terminal insulating member, the device being configured so that particulate matter can be electrically adsorbed on the wall surface of the through-hole, and this particulate matter can be detected by measuring a change in electrical properties of the wall that defines the through-hole.

Although the device of the '632 publication may be adequate for some applications, it may be less than optimal. For example, the device may be prone to malfunctions under saturation conditions, when the wall surface of the through hole becomes completely covered with particulate matter. Further, the wall surface may require significant maintenance to remove excess particulate matter during the saturation conditions.

The given method of the present disclosure addresses one or more of the problems set forth above and/or other problems of the prior art.

### SUMMARY

In one aspect, the present disclosure is related to a method of monitoring a gas. The gas monitoring method may include applying a voltage pulse to electrodes exposed to the gas, wherein the voltage pulse has a higher voltage amplitude than a breakdown voltage of the gas. The method may also include detecting breakdown of the gas, and determining a time to breakdown of the gas since application of the voltage pulse. The method may additionally include determining a concentration of particulate matter entrained in the gas based on the time to breakdown.

In another aspect, the present disclosure is related to another gas monitoring method. This gas monitoring method may include applying a series of voltage pulses to electrodes exposed to the gas, wherein each subsequent voltage pulse in the series of voltage pulses has an incrementally higher voltage amplitude than a preceding voltage pulse in the series of voltage pulses. The method may also include detecting breakdown of the gas during application of one of the series of voltage pulses, and determining a concentration of particulate matter entrained in the gas based on a voltage amplitude of the one of the series of voltage pulses.

In another aspect, the present disclosure is related to yet another gas monitoring method. This method may include applying a first voltage pulse to upstream electrodes exposed to the gas to charge particulate matter in the gas, and applying a second voltage pulse to downstream electrodes exposed to the gas to further charge the particulate matter. The method may also include monitoring a current at the downstream electrodes during discharge of the particulate matter, and determining a concentration of particulate matter entrained in the gas based on a value of the current.

### BRIEF DESCRIPTION OF THE DRAWINGS

FIG. 1 is a diagrammatic illustration of an exemplary disclosed power system;

FIG. 2 is a diagrammatic illustration of an exemplary disclosed gas monitoring system that may be used in conjunction with the power system of FIG. 1;

FIGS. 3-6 are a schematic and diagrammatic illustrations of various electrode configurations that form a portion of the gas monitoring system of FIG. 2; and

FIG. 7 is another exemplary disclosed gas monitoring system that may be used in conjunction with the power system of FIG. 1.

### DETAILED DESCRIPTION

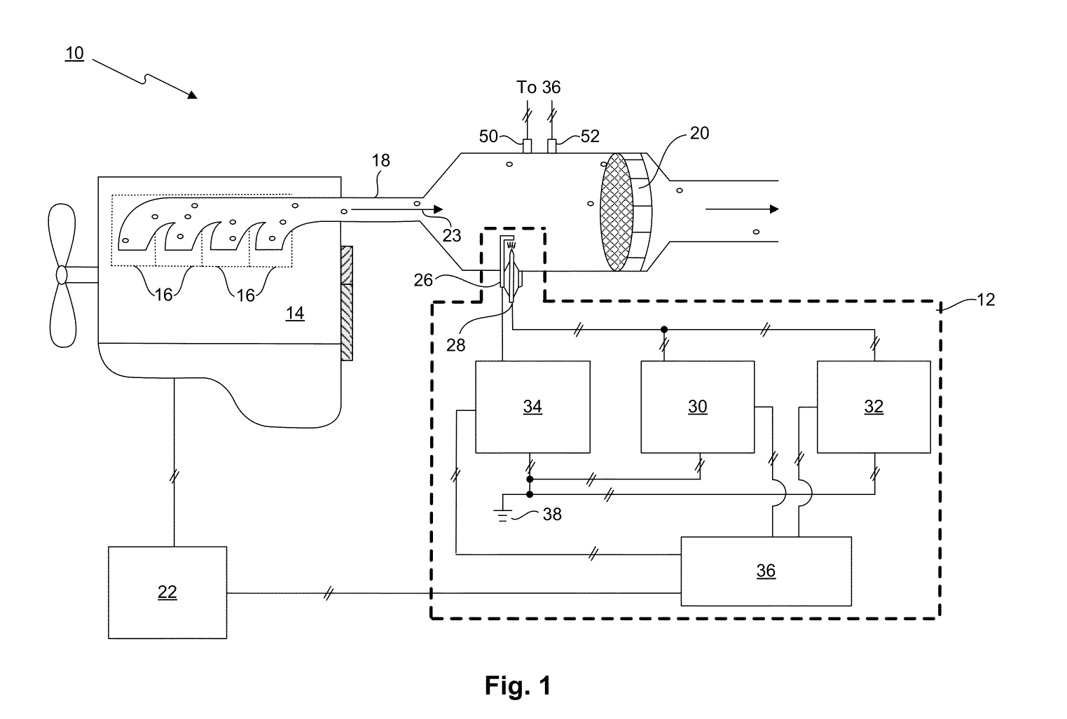

FIG. 1 illustrates an exemplary power system 10 incorporating a soot detection system 12 consistent with this disclosure. For the purposes of this disclosure, power system 10 is depicted and described as an internal combustion engine, for example a gasoline, diesel, or gaseous fuel-powered engine that draws in a flow of combustion gases and produces a flow of exhaust gas 23. However, it is contemplated that power system 10 may embody any other type of gas producing, treating, and/or handling system known in the art where detection of non-gaseous matter entrained within the associated gas (e.g., particulate matter) is desired.

Power system 10, as an internal combustion engine, may include an engine block 14 that at least partially defines a plurality of cylinders 16, and a plurality of piston assemblies (not shown) disposed within cylinders 16. Cylinders 16, together with the pistons, may form a plurality of combustion chambers. It is contemplated that power system 10 may include any number of combustion chambers and that the combustion chambers may be disposed in an “in-line” configuration, a “V” configuration, or in any other conventional configuration. An exhaust passage 18 may extend from the combustion chambers to the atmosphere, and one or more different treatment devices 20 (e.g., particulate filters, reductant injectors, catalysts, attenuation devices, etc.) may be disposed within exhaust passage 18.

In some embodiments, power system 10 may be equipped with a general system controller 22. In these embodiments, system controller 22 may be configured to regulate operations of power system 10, for example fuel injection, boosting, gas mixing, valve timing, exhaust gas recirculation, reductant dosing, and other operations, to affect production of particulate matter and/or its discharge to the atmosphere.

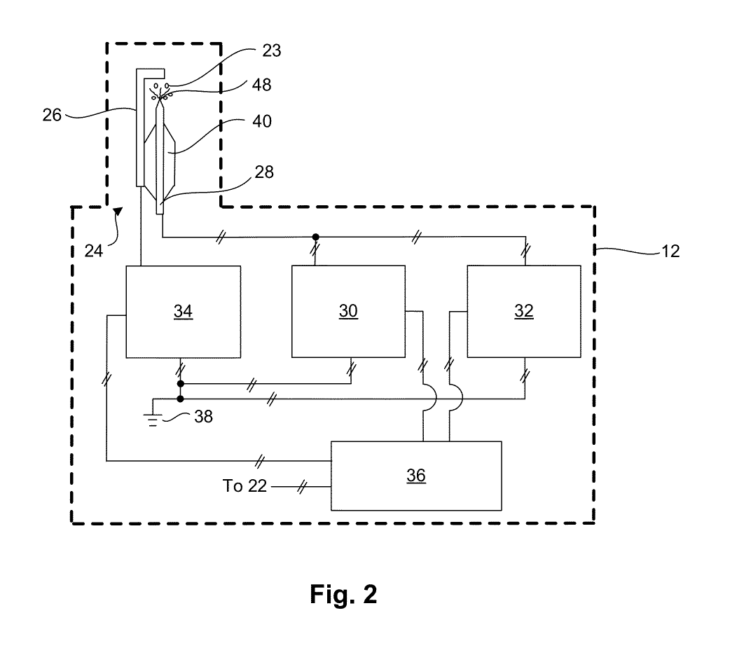

As shown in FIG. 2, soot detection system 12 may include components that cooperate to determine a concentration of particulate matter within the exhaust gas 23 of power system 10 flowing through exhaust passage 18. The concentration information may then be utilized by system controller 22 to help regulate the different operations of power system 10. Soot detection system 12 may include, among other things, electrodes 24 (including at least one anode 26 and at least one cathode 28), a pulse generator 30, a voltage measurement device 32, a current measurement device 34, and a detection controller 36. Electrodes 24 may be positioned in fluid communication with the exhaust gas 23 of exhaust passage 18 such that a discharge path between anode 26 and cathode 28 may be created within the exhaust gas 23. Pulse generator 30, voltage measurement device 32, current measurement device 34, and detection controller 36 may be located anywhere onboard or in the immediate proximity to power system 10, and be in communication with each other, with electrodes 24, and/or with system controller 22.

Anode 26 may embody a conductive element, for example an element composed of carbon nanotubes, carbon fibers, stainless or non-stainless steel, tantalum, platinum, tungsten, silver, gold, high-nickel alloys, copper, or other conductive elements. During normal operation (e.g., when a negative voltage is applied to electrodes 24) anode 26 may be connected to an electrical ground 38, such as an earth ground, or other ground. In other operations (e.g., when a positive voltage is applied to electrodes 24), the anode 26 may be insulated from ground 38 via an insulator 40.

Cathode 28 may also embody a conductive element substantially similar to anode 26. However, cathode 28, in contrast to anode 26 may be insulated from ground 38 via insulator 40 during normal operations, and connected to ground 38 during the other operations. Additionally, depending on the particular geometry of cathode 28 and/or anode 26, it may be necessary to insulate portions of cathode 28 from anode 26. Insulator 40 may include, for example, a material fabricated from aluminum oxide, aluminum nitride, porcelain, boron nitride, or other insulating elements.

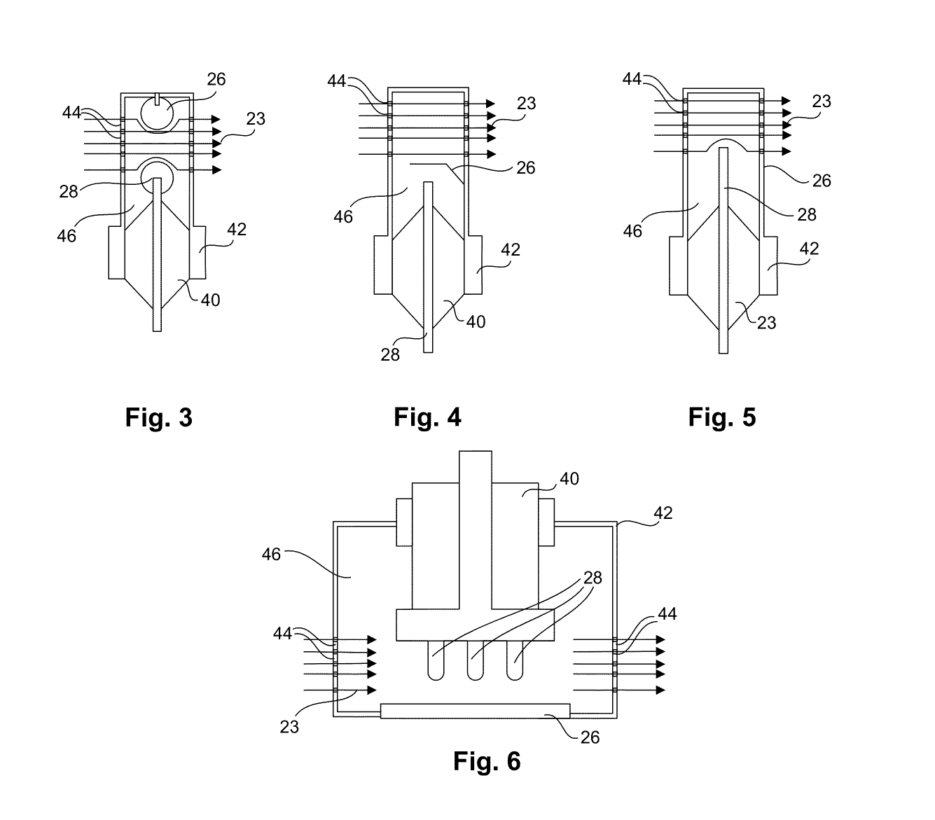

The configuration of electrodes 24 shown in FIG. 1 is known as a point-to-plane configuration. In this configuration, cathode 28 may come to a point and anode 26 may be generally planar and spaced apart from cathode 28 in an orthogonal orientation, such that a discharge of electricity may be possible from the point of cathode 28 to any location on anode 26. It should be noted, however, that many other electrode configurations are also possible. For example, FIG. 3 illustrates anode 26 and cathode 28 as generally spherical conductors. In this configuration, anode 26 may be electrically and mechanically coupled to an anode cap 42 that substantially surrounds cathode 28. Anode cap 42 may have a plurality of openings 44 that allow the exhaust gas 23 from power system 10 to pass through a discharge space 46 between anode 26 and cathode 28. In the configuration of FIG. 4, cathode 28 may be a generally cylindrical conductor, and anode 26 may be a conductor that is positioned generally perpendicular to cathode 28. FIG. 5 illustrates cathode 28 as being a generally cylindrical conductor located within anode cap 42, and anode 26 as being generally integral with anode cap 42 and coaxial to cathode 28. In this configuration, the discharge path between electrodes 24 may occur radially outward from cathode 28 to anode 26. Finally, FIG. 6 illustrates an electrode configuration having a multiple point-type cathode 28 that interacts with a single generally planar anode 26, which is generally perpendicular to cathode 28. The configuration of FIG. 6 may be capable of creating a multi-point discharge within the exhaust gas 23 between anode 26 and cathode 28. Additionally, in some embodiments (not shown), a dielectric may be located within the discharge path between cathode 28 and anode 26, for example as a coating on anode 26 and/or cathode 28.

Referring back to FIG. 2, the configuration of pulse generator 30 may be based on a capacitive architecture, an inductive architecture, or a combination thereof. A capacitive-based architecture may include of one or more capacitors disposed in series (e.g., a capacitor bank) or in parallel (e.g., a Marx bank). An inductive-based architecture may include one or more magnetic inductors such as an induction coil also known as an inductive adder. A combination capacitive-inductive architecture may include both inductive and capacitive components coupled to function together through the use of magnetic compression. Additionally, in some embodiments, pulse generator 30 may use one or more transmission lines (e.g., a Blumlien), if desired. Pulse generator 30 may be a stand-alone component (shown in FIG. 2) or, alternatively, form an integral part of detection controller 36, as desired.

Pulse generator 30 may include or be connected to a source of electrical power (not shown). In one example, pulse generator 30 may include an integral energy storage device that functions as the source of electrical power. In another example, the energy storage device may be a separate unit, for example, a bank of one or more capacitors, a bank of one or more inductors, or a combination thereof. The energy storage device, in these embodiments, may be charged by a separate supply voltage (e.g., the voltage from an power system battery, a rectified utility voltage, etc.).

Pulse generator 30 may be controlled to generate and apply one or more voltage pulses to electrodes 24 to cause a discharge between cathode 28 and anode 26 that creates a plasma 48 in the exhaust gas 23 of power system 10 (to cause breakdown of a constituent in the exhaust gas 23). In some embodiments, pulse generator 30 may be capable of producing a continuous train of discrete pulses, such as negative voltage pulses. However, it is contemplated that pulse generator 30 may additionally or alternatively be configured to create one or more positive voltage pulses, as desired.

The output of pulse generator 30 may be adjusted to help generate either a thermal plasma (e.g., an arc) or non-thermal plasma between electrodes 24 during discharge. In particular, one or more of a width, an amplitude, and a frequency of the pulse created by pulse generator 30 may be selectively adjusted by detection controller 36 to thereby control characteristics of the resulting plasma 48. For example, the pulse width may be varied within a range of about 1-10 μs, while the pulse amplitude may be varied within a range of about 0.5-20 kV. Similarly, the pulse frequency may range from a single pulse to frequencies in the kHz. Although a thermal plasma may be helpful in some situations for charging of soot particles, prevention of a thermal plasma between electrodes 24 in other situations may help to reduce electrode erosion and energy supply requirements of soot detection system 12.

Voltage measurement device 32 may embody a voltage divider, for example a resistive or capacitive voltage divider, that is configured to measure an actual voltage across discharge space 46. Voltage measurement device 32 may be configured to generate a voltage signal indicative of the actual voltage and direct the voltage signal to detection controller 36 for further processing. It is contemplated that voltage measurement device 32 may additionally be configured to provide the voltage signal to another system or device, for example, to system controller 22 (referring to FIG. 1), to an oscilloscope, to an offboard computer, etc., if desired.

Current measurement device 34 may embody a current transformer configured to measure an actual current between electrodes 24 during discharge. Current measurement device 34 may be further configured to generate a current signal indicative of the actual current and direct the current signal to controller 36 for further processing. It is contemplated that current measurement device 34 may additionally be configured to provide the current signal to another system or device, for example, to system controller 22 (referring to FIG. 1), to an oscilloscope, to an offboard computer, etc., as desired.

Detection controller 36 may include a processor (not shown), a memory (not shown), and/or a data interface (not shown). The processor(s) may be a single or multiple microprocessors, field programmable gate arrays (FPGAs), or digital signal processors (DSPs) capable of executing particular sets of instructions. The instructions executed by the processor may be pre-loaded into the processor or may be stored in separate computer-readable memory (not shown) or other separate storage device (not shown), such as a random access memory (RAM), a read-only memory (ROM), a hard disk, an optical disk, a magnetic medium, a flash memory, other permanent memory, other volatile memory, or any other tangible mechanism capable of providing instructions to the processor. Additionally, one or more lookup tables (not shown) may be stored in the processor and/or separate computer-readable memory, as desired, and referenced by the processor during execution of the instructions.

It should be appreciated that detection controller 36 could be dedicated to only soot detection functions or, alternatively, integral with general system controller 22 (referring to FIG. 1) and be capable of controlling numerous power system functions and modes of operation. If separate from system controller 22, detection controller 36 may communicate with system controller 22 via data links or other methods. Various other known circuits may be associated with detection controller 36, including power supply circuitry, signal-conditioning circuitry, actuator driver circuitry (i.e., circuitry powering solenoids, motors, or piezo actuators), communication circuitry, and other appropriate circuitry. In some embodiments, detection controller 36 may be coupled to input/output devices (e.g., to a monitor, a keyboard, a printer, etc.) to receive input from a user and output information to the user. Detection controller 36 may be configured to communicate with other systems and/or devices, for example, an oscilloscope, a computer, etc., as desired. Additionally, in some embodiments, detection controller 36 may be configured to send control signals or otherwise communicate with one or all of pulse generator 30, voltage measurement device 32, current measurement device 34, and electrodes 24.

The lookup table used by detection controller 36 may contain information helpful in determining a concentration of soot entrained within exhaust gas 23. For example, the lookup table may include voltage values associated with breakdown of the exhaust gas 23 for different concentrations of particulate matter, and time durations required for the breakdown events to occur after the exhaust gas 23 is first exposed to a known voltage pulse under particular conditions. Under normal conditions (i.e., when a voltage pulse is not applied to electrodes 24), the exhaust gas 23 between anode 26 and cathode 28 may function as an insulator, preventing electricity from being conducted therebetween. However, a period of time after a known pulse of electrical energy having a sufficiently high voltage is first applied to electrodes 24 (i.e., a period of time after a voltage exceeding a dielectric strength of constituents in the exhaust gas 23 is first applied to electrodes 24), the exhaust gas 23 between electrodes 24 may “break down” or partially ionize and function as a conductor to conduct the energy from cathode 28 to anode 26. The exhaust gas 23 may break down when exposed to different levels of voltage, depending on the concentration of particulate matter within the exhaust gas 23. Similarly, the exhaust gas 23 may break down after a different period of time has elapsed following application of the voltage pulse, the elapsed period of time relating to the concentration of particulate matter in the exhaust gas. The lookup table may store these different voltage values and time durations, along with the corresponding concentrations of soot and the conditions underwhich breakdown events occur. Measured values of the voltage pulse that cause breakdown of the exhaust gas 23 and/or a tracked amount of time to breakdown following application of the known voltage pulse may then be referenced by detection controller 36 with the lookup table to identify the concentration of particulate matter in the exhaust gas 23. An example of this operation will be provided in the following section of this disclosure.

It is contemplated that the lookup table may alternatively or additionally contain information relating a current spike measured during discharge of previously-charged particulate matter to the concentration of particulate matter within the exhaust gas 23. In particular, as will be described below, it may be possible to charge the particulate matter and then measure a spike in current that occurs at a time of discharge. Detection controller 36 may then be configured to reference a value of the measured current spike with the lookup table to determine the concentration of particulate matter. An example of this operation will also be provided in the following section of this disclosure.

For the purposes of this disclosure, a spike in voltage or current may refer to a characteristic of a measured voltage or current, where the measured voltage or current rapidly increases for a brief period of time, beyond an amplitude normally expected during and after an applied voltage pulse, and then rapidly decrease back to the expected amplitude. The spike may occur for only a very short amount of time, e.g., less than a micro second.

One or more parameter sensors may be associated with detection controller 36 to facilitate determination of the particulate matter concentration within the exhaust gas 23 of power system 10. For example, a temperature sensor 50 and/or a pressure sensor 52 may be disposed in fluid communication with the exhaust gas 23 of exhaust passage 18 at locations near electrodes 24, and be configured to generate corresponding signals directed to detection controller 36. Detection controller 36 may be configured to determine the current conditions (e.g., temperatures and/or pressures of the exhaust gas 23) based on the signals, and affect use of the lookup tables accordingly. It is contemplated that the current conditions may alternatively be calculated from other measured parameters, instead of being directly measured, if desired. It is further considered that other parameters, for example a humidity of the exhaust gas 23, may alternatively be sensed and utilized to affect use of the lookup tables, if desired.

Detection controller 36 may regulate operation of pulse generator 30 to selectively generate a voltage pulse having particular characteristics. In particular, detection controller 36 may be configured to dynamically adjust a voltage, a width, and/or a frequency of the pulse generated by pulse generator 30. Alternatively, detection controller 36 may be configured to simply trigger pulse generator 30 to generate one or more pre-determined voltage pulses. Detection controller 36 may then reference signals from voltage measurement device 32 and/or current measurement device 34 at the time of constituent breakdown, along with the elapsed period of time since application of the voltage pulse, with the lookup tables to determine the concentration of particulate matter. In some situations, detection controller 36 may benefit from noise reduction and/or filtering on the voltage and current signals during the analysis. Additionally, detection controller 36 may be configured to trend changes in particulate matter concentration over time and/or under different operating conditions of power system 10, as desired.

It is contemplated that detection controller 36 may take specific corrective actions in response to detection of particulate matter concentrations that exceed threshold levels during comparison by detection controller 36. The corrective actions may include, for example, making adjustments to the operation of power system 10 via system controller 22, activation of alarms or alerts, regulation of gas mixing, and other actions known in the art.

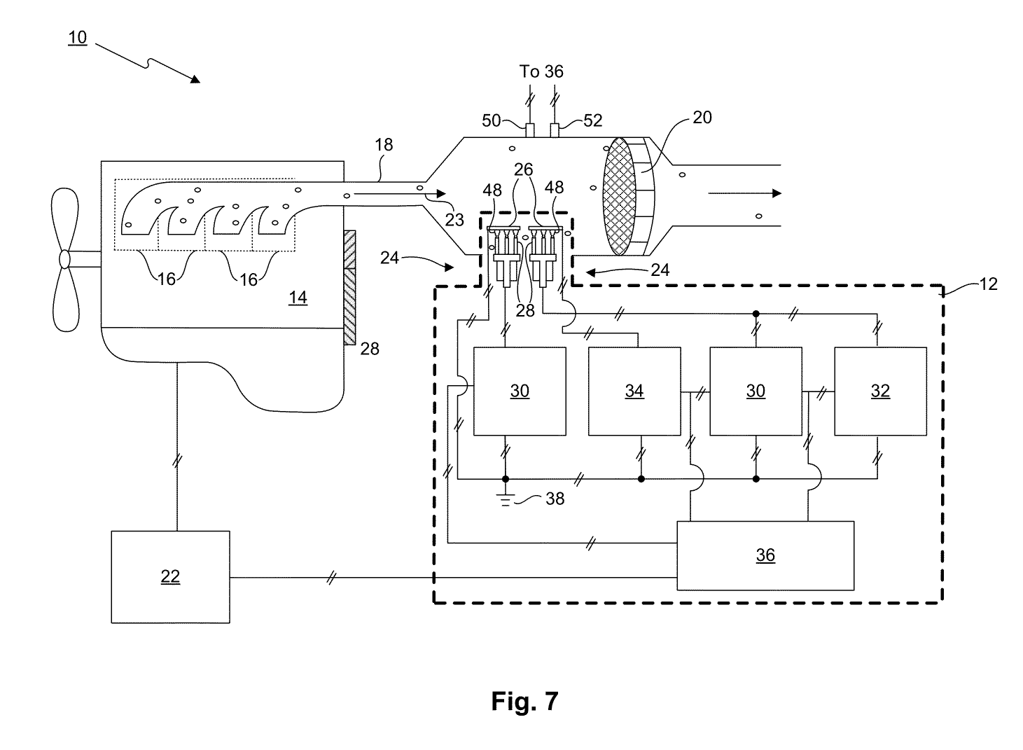

FIG. 7 illustrates an alternative embodiment of soot detection system 12. Similar to the embodiment of FIGS. 1 and 2, soot detection system 12 of FIG. 7 may include electrodes 24, pulse generator 30, voltage measurement device 32, current measurement device 34, and detection controller 36. However, in contrast to the embodiment of FIGS. 1 and 2, soot detection system 12 of FIG. 7 may include an additional pair of electrodes 24 located upstream from the existing electrodes 24, for a total of at least two pairs of substantially identical electrodes 24 located in series along a flow path of the exhaust gas 23. In addition, electrodes 24 of FIG. 7 may be of the multi-point type. The soot detection system 12 of FIG. 7 may also include an additional pulse generator 30 associated with the upstream electrodes 24. In this configuration, voltage and current measuring devices 32, 34 may only be associated with the downstream electrodes 24. It is contemplated, however, that soot detection system 12 of FIG. 7 may alternatively include an additional voltage and/or current measure device 32, 34 associated with the upstream electrodes 24 for diagnostic purposes, if desired.

### INDUSTRIAL APPLICABILITY

The soot detection system of the present disclosure may be used in any application where it is desired to determine a concentration of particulate matter within a gas. The soot detection system may determine the concentration of particulate matter within the gas by selectively applying voltage pulses to electrodes 24, and measuring characteristics of resulting discharges. The characteristics may then be referenced with a calibrated lookup table to determine the concentration. Potential applications for the disclosed soot detection system include, among others, engine system or furnace applications. Operation of soot detection system 12 will now be described in detail.

During operation of the soot detection system 12 depicted in FIG. 1, detection controller 36 may cause pulse generator 30 to generate and apply one or more voltage pulses to electrodes 24, thereby creating a thermal plasma 48 (i.e., an arc) between electrodes 24. The voltage pulse may have an amplitude of V_{A}. When V_{A} is greater than the breakdown voltage of the exhaust gas 23, arcing between electrodes 24 may occur. As the concentration of particulate matter in the exhaust gas 23 increases, the V_{A} required to cause arcing and/or the time duration required for a given voltage pulse to cause arcing may decrease. Accordingly, detection controller 36, of the embodiment shown in FIG. 1, may determine particulate matter concentration in two different ways. First, controller 36 may cause pulse generator 30 to generate and apply a series of voltage pulses to electrodes 24, each subsequent pulse in the series having an incrementally greater V_{A}, until thermal discharge occurs, and then reference the V_{A} that caused the discharge with the lookup table to determine the corresponding concentration of particulate matter. Thermal discharge may be considered to have occurred when a voltage between electrodes 24 drops (as measured by voltage measuring device 32) and a current between electrodes 24 sharply increases and then decreases (i.e., spikes, as measured by current measuring device 34). Second, controller 36 may cause pulse generator 30 to generate and apply a voltage pulse known to have a V_{A} higher than required to cause the discharge of the exhaust gas 23, and reference the V_{A} and an elapsed time from application of the pulse to discharge with the lookup table to determine the corresponding concentration.

During operation of the soot detection system 12 depicted in FIG. 7, detection controller 36 may cause pulse generator 30 associated with the upstream-located electrodes 24 to generate and apply one or more voltage pulses to electrodes 24, thereby creating a thermal plasma 48 (i.e., an arc) between each distal point of the multi-point cathode 28 and the planar anode 26. In this configuration, the thermal plasma 48 may encompass a much larger area and/or a greater amount of particulate-laden exhaust gas 23, as compared with a single point-to-plane electrode configuration. When thermal plasma 48 is generated within the exhaust gas 23 at the upstream electrodes 24, electrons associated with the thermal plasma 48 may charge particulate matter entrained within the exhaust gas 23. As the exhaust gas 23, having particulate matter now somewhat charged from the first voltage pulse, reaches the downstream electrodes 24, detection controller 36 may cause pulse generator 30 associated with the downstream-located electrodes 24 to generate and apply one or more additional voltage pulses to the downstream electrodes 24, thereby creating a non-thermal plasma 48 between each distal point of the multi-point cathode 28 and the planar anode 26. The non-thermal plasma 48 may function to further charge the particulate matter. A period of time after cessation of the second voltage pulse, the charged particulates may begin to discharge to anode 26. This discharge may start slowly, reach a maximum, and the slow down again, resulting a measurable spike in current from the particulate matter to anode 26. Current measuring devices 34 may detect this spike in current, and generate a signal indicative of a value of the spike directed to detection controller 36. Detection controller 36 may reference the value of the current spike with the lookup table to determine a corresponding concentration of particulate matter entrained with the exhaust gas 23. As the concentration of particulate matter increases, the value of the spike may generally tend to decrease.

Soot detection system 12 of FIG. 7 may utilize the first voltage pulse to generate the thermal plasma and the second voltage pulse to generate the non-thermal plasma for several reasons. First, a single voltage pulse that generates either a non-thermal plasma or a thermal plasma, by itself, may impart too little charge to the particulate matter to be detectable via voltage and/or current measuring devices 32, 34. Second, two pulses that both generate a thermal plasma could result in premature erosion of electrodes 24, while also consuming larger amounts of energy. Accordingly, a combination of the first pulse to generate a thermal plasma and the second pulse to generate a non-thermal plasma may result in sufficient charging of the particulate matter, while providing for longevity of electrodes 24 and reducing energy consumption of soot detection system 12. It is contemplated, however, that the first pulse may alternatively generate the non-thermal plasma and the second pulse may generate the thermal plasma, if desired.

In some situations, the lookup tables may not have data corresponding to the measured voltage and/or current values, the time durations, the electrode configuration, and/or the parameters of the exhaust gas 23. In these situations, any measured voltage or current spike(s) may be determined to be caused by electrical noise or an unexpected condition within the exhaust gas. When this occurs, detection controller 36 may notify a user of soot detection system 12 of the anomalous result.

In some embodiments, the measured voltage and/or current spike values may be stored in a buffer between the applications of subsequent voltage pulses, which may occur, for example, at a repetition frequency between about 50 kHz to 60 kHz. The measured voltage and/or current spike values may be stored until some threshold is met within the buffer (e.g., data from 1000 pulses may be stored in the buffer). Once the threshold is met, detection controller 36 may perform error reduction on the measured data in the buffer, before determining the concentration of particulate matter. For example, detection controller 36 may average the buffer values for the voltage spikes, the current spikes, and/or the time durations. Detection controller 36 may then use the averaged values to determine the concentration of particulate matter. Detection controller 36 may maintain a first-in-first-out queue, such that the average buffer data is continually being updated. Alternatively, detection controller 36 could process the buffer values in blocks. For example, detection controller may average the first 1000 values and then wait until the buffer fills again to process the next 1000 values, etc.

Several advantages may be associated with soot detection system 12. For example, soot detection system 12 may be capable of rapidly determining a concentration of particulate matter in a gas using short, low-voltage pulses, which may help to reduce energy consumption. Moreover, the plasma created between electrodes 24 in the configuration of FIG. 7, during the second pulse, may be a non-thermal plasma, which may help to reduce potential electrode erosion (i.e., as compared to two consecutive thermal-plasma events). Additionally, by not using a dielectric barrier between the electrodes (e.g., dielectric barrier discharge) the method may be more robust in high vibration environments. Finally, in the configuration of FIG. 7, by directly measuring the ion current of the charged soot particles measurement error may be reduced, in particular, when compared to methods that add additional steps to infer the ion current of the soot particles.

It will be apparent to those skilled in the art that various modifications and variations can be made to the methods of the present disclosure without departing from the scope of the disclosure. Other embodiments will be apparent to those skilled in the art from consideration of the specification and practice of the methods disclosed herein. It is intended that the specification and examples be considered as exemplary only, with a true scope of the disclosure being indicated by the following claims and their equivalents.

What is claimed is:

1. A method for monitoring a gas, comprising:
applying a voltage pulse to electrodes exposed to the gas, wherein the voltage pulse has a higher voltage amplitude than a breakdown voltage of the gas;
detecting breakdown of the gas;
determining a time to breakdown of the gas since application of the voltage pulse; and
determining a concentration of particulate matter entrained in the gas based on the time to breakdown.

2. The method of claim 1, wherein detecting breakdown of the gas includes detecting a thermal discharge at the electrodes.

3. The method of claim 1, wherein the time to breakdown decreases with an increasing concentration of particulate matter.

4. The method of claim 1, wherein the electrodes have a point-to-plane configuration.

5. The method of claim 1, further including taking corrective action when the concentration of particulate matter is determined to exceed a threshold.

6. The method of claim 5, wherein:
the gas is exhaust gas from an engine; and
taking corrective action includes adjusting operation of the engine to reduce production of particulate matter or increase treatment of particulate matter.

7. A method for monitoring a gas, comprising:
applying a series of voltage pulses to electrodes exposed to the gas, wherein each subsequent voltage pulse in the series of voltage pulses has an incrementally higher voltage amplitude than a preceding voltage pulse in the series of voltage pulses;
detecting breakdown of the gas during application of one of the series of voltage pulses; and
determining a concentration of particulate matter entrained in the gas based on a voltage amplitude of the one of the series of voltage pulses.

8. The method of claim 7, wherein detecting breakdown of the gas includes detecting a thermal discharge at the electrodes.

9. The method of claim 7, wherein a voltage amplitude required to breakdown the gas decreases with increasing concentration of particulate matter.

10. The method of claim 7, wherein the electrodes have a point-to-plane configuration.

11. The method of claim 7, further including taking corrective action when the concentration of particulate matter is determined to exceed a threshold.

12. The method of claim 11, wherein:
the gas is exhaust gas from an engine; and
taking corrective action includes adjusting operation of the engine to reduce production of particulate matter or increase treatment of particulate matter.

13. A method for monitoring a gas, comprising:
applying a first voltage pulse to upstream electrodes exposed to the gas to charge particulate matter in the gas;
applying a second voltage pulse to downstream electrodes exposed to the gas to further charge the particulate matter;
monitoring a current at the downstream electrodes during discharge of the particulate matter; and
determining a concentration of particulate matter entrained in the gas based on a value of the current.

14. The method of claim 13, wherein:
the current at the downstream electrodes spikes after cessation of the second voltage pulse; and
determining the concentration of particulate matter includes determining the concentration of particulate matter based on a value of the spike.

15. The method of claim 14, wherein the value of the spike increases with increasing concentration of particulate matter.

16. The method of claim 13, wherein the downstream electrodes have a multi-point configuration.

17. The method of claim 16, wherein the upstream electrodes are substantially identical to the downstream electrodes.

18. The method of claim 13, further including taking corrective action when the concentration of particulate matter is determined to exceed a threshold.

19. The method of claim 18, wherein:
the gas is exhaust gas from an engine; and
taking corrective action includes adjusting operation of the engine to reduce production of particulate matter or increase treatment of particulate matter.

20. The method of claim 13, wherein:
one of the first and second pulses generates a non-thermal plasma in the gas; and
the other of the first and second pulses generates a thermal plasma in the gas.
